(19) United States
(12) Patent Application Publication
(10) Pub. No.: US 2008/0317730 A1
(43) Pub. Date: Dec. 25, 2008

(54) Compositions Useful for Recovering Original Trophism and Pigmentation, and for Stimulating Growth of the Integumentary Apparatus, Uses and Products Thereof

(76) Inventors: Luisa Gennero, Torino (IT); Antonio Ponzetto, Moncalieri (Torino) (IT); Enrico De Vivo, Venaria Reale (Torino) (IT); Luciano Contu, Alice Castello (Vercelli) (IT); Emanuella Morra, Torino (IT); Chiara Cesano, Torino (IT)

Correspondence Address: Steinfl & Bruno, 301 N Lake Ave Ste 810, Pasadena, CA, 91101, US

(21) Appl. No.: 12/093,112

(22) PCT Filed: Nov. 10, 2006
(86) PCT No.: PCT/IB2006/054193

§ 371 Date: May 8, 2008

(30) Foreign Application Priority Data
Nov. 11, 2005 (IT) TO2005000800

(51) Int. Cl. A61K 38/45; A61K 38/43; A61K 38/48; A61P 17/00; A61K 38/46

(52) U.S. Cl. 424/94.5; 424/94.1; 424/94.65; 424/94.67; 424/94.63; 424/94.6; 424/94.64

(57) ABSTRACT

The present invention relates to compositions and products comprising at least one proteolytic enzyme adapted to promoting the restoration of original trophism of the integumentary apparatus, the original pigmentation and stimulating the growth thereof and, particularly, improving the viability of the pilifer follicles, even in scar tissue areas.

The present invention relates to compositions and products adapted to promoting the recovery of the original trophism of the integumentary apparatus, the original pigmentation thereof and stimulating the growth thereof and, particularly, improving the viability of the pilifer follicles, even in scar tissue areas.

Accordingly, the present invention relates to compositions and products capable of preferably being used in the treatment of alopecia, defluvium and effluvium, in stem regeneration, in original hair pigmentation and in restoring the trophism of the integumentary apparatus.

### TECHNICAL BACKGROUND OF THE INVENTION

By the term alopecia is meant the absence or lack of hair in areas of the skin where they are normally found. The term alopecia also comprises both hypotrichia, indicating the lack of hair, and “baldness”, which indicates irreversible hair loss.

On the other hand, the term “defluvium” is used when indicating hair loss that is abnormal in terms of quantity and quality, while “effluvium” should only be used in cases where hair , loss is numerically very high, even several hundred hairs each day, and qualitatively homogeneous.

Classically, alopecia is distinguished as being temporary (transient functional inhibition of the hair papilla) and definitive (disappearance of the follicle and the germinative papilla, or root). A distinction must be made between these and pseudo-alopecia, where the hair has been torn out or broken (trichoclasia) as a result of trauma, chemical action, infection or congenital anomalies of the stem.

Alopecia can be the result of genetic factors, ageing and local or systemic disorders. Seborrheic dermatitis and psoriasis are the disorders most commonly affecting the scalp, but very rarely cause alopecia. Alopecia may be the result of scarring or not, toxic-medicative, Brocq's alopecia areata or pseudoareata, iatrogen (generally caused by medications), post-natal, post-infective, and may also be caused by trichotillomania, ringworm, kerion formation and tinea favosa. Alopecia can also be caused by lupus erythematosus (in both systemic and fixed discoid form), schleroderma, lychen planus, follicular mucinosis or decalcifying folliculitis, as well as aplasia cutis (loss of skin) or tumours [1-4].

Androgenic alopecia only appears if the concentrations of male hormones reach the levels found in adults, and is therefore never observed prior to puberty. Baldness in men does not depend on excess androgens, but on an excessive response of the integumentary apparatus to said hormones [5].

The sensitivity of the hair, or to be more precise the pilifer follicles, to androgens, depends mainly on a particular enzyme, type-II 5-alpha-reductase, produced by follicle cells [5]. This enzyme transforms testosterone, the main male hormone, into its more potent derivative, dihydrotestosterone or DHT, the main agent responsible for androgenic alopecia. Indeed, the follicles of those areas on the scalp affected by baldness produce high quantities of this enzyme, and hence high quantities of DHT [5].

Androgenic baldness in women generally appears around the age of thirty five and is typically manifested in three stages. Above all in young women, thinning is frequently more evident at the forehead [4-5]. On the other hand, in menopausal women a receding hairline, similar to that seen in men, is frequently observed. However, even in the most serious cases complete baldness is never observed, only serious thinning [4-5]. In women, androgenic baldness may be the result of excess male hormones or excessive sensitivity of the integumentary apparatus to normal levels of androgens [4-5].

There have been numerous studies conducted on hair growth in general and head hair in particular, such as the attempts to develop compositions which might resolve the problems of alopecia, effluvium or defluvium in humans. Among the various studies, it is worthwhile mentioning the work of Robinson M et al. [6], in relation to the in vitro development of the vibrisse follicles in adult rats. This work has formed the basis for defining a culture protocol capable of maintaining the viability of the pilifer follicles for over 20 days (prior to this work, “in vitro” hair growth generally stopped prematurely in comparison to the situation observed “in vivo”) [6]. Microscopic examination has shown that, despite widespread pathological changes in the epithelium of the follicle, the follicle cells showed a significant capacity for recovery. Hence, this data confirms that hair loss is not the result of the loss of follicle regenerative capacity (which in any case remain living in a state of quiescence or, even better, reversible atrophy), but on a collection of factors which condition the life-cycle of the follicle itself [1-6].

### DESCRIPTION OF THE INVENTION

An object of the present invention is that of finding a valid and effective solution for combating the problems of hair loss and changes in the integumentary apparatus in mammals, and humans in particular, which have remained unresolved to date.

A further object of the present invention is that of finding a valid solution for promoting the recovery of the original pigmentation and the original trophism of the integumentary apparatus, for example by promoting greater hair stem diameter or the reduced desquamation thereof.

According to the present invention, these objects are achieved thanks to the solution specifically claimed in the appended claims. The claims are an integral part of the technical teaching provided in relation to the invention.

The invention relates to compositions and products with the capacity to promote the recovery of original trophism and the original pigmentation of the integumentary apparatus, as well as stimulating the growth of the integumentary apparatus itself and, particularly, improving the viability of the pilifer follicles.

The present invention is based on the observation of a special proliferative stimulus exercised by certain proteolytic agents on the pilifer follicles included in certain biopsy samples which were investigated within the frame of the present research. Said observations have lead to the formulation of a composition useful for stimulating hair growth and/or improving viability of the atrophic or non atrophic pilifer follicle. After twenty-one days of treatment in vitro with the composition being the object of the present invention, all types of hair, new and otherwise, recover their original trophism, appear reinvigorated, with original pigmentation and furthermore, have increased healthy stem diameter without any desquamation.

The composition being the object of the present invention (referred to hereinafter as FORM-XE) is capable of stimulating hair growth by means of direct action on the pilifer follicles with the recovery of atrophic follicles; even scar tissue areas are repopulated with active pilifer follicles.

The invention will now be described in detail merely by way of a non-limiting example.

According to the present inventors, the combined use of at least one proteolytic enzyme in association with at least one aminoacid and/or one vitamin (or vitamin factor) results in more complete action on the mechanisms of hair regrowth, or rather the stimulation of the pilifer bulbs in order to restore the original activity thereof.

Indeed, the present inventors have found that the proteolytic enzyme, in combination with at least one of the aminoacids and one vitamin or vitamin factor, and preferably with a mucopolysaccharide or a nucleotide or a nucleoside or a sugar (monosaccharide or polysaccharide) unexpectedly results in the potent and entirely positive stimulation of the growth of the integumentary apparatus, as well as the recovery of the original trophism and pigmentation of compromised integumentary apparatus. This result is unexpected, considering that compositions containing proteolytic enzymes are known to cause hair loss and are used as depilatory treatments.

The composition being the object of the present invention comprises at least one proteolytic enzyme (for example papain) and at least one substance useful for nourishing the follicle, preferably at least one aminoacid or one vitamin or vitamin factor, preferably a vitamin with reducing activity. Furthermore, other components which play the role of in vivo growth factors, including glucose, may also be present. The composition being the object of the present invention may further contain a mucopolysaccharide, such as for example hyaluronic acid. The components of the composition are suspended in a hydroalcoholic solution, preferably including 0.2% minoxidil, where the use of minoxidil has the sole purpose of improving the permeability of the scalp by stimulating blood circulation [7] or Transcutol CG (ethoxydiglycol) with the same aim of improving scalp permeability.

Without wishing to be bound to any specific theory, the present inventors maintain that the proteolytic enzyme, optionally in association with at least one of the aminoacids, one vitamin or vitamin factor and optionally one nucleotide or nucleoside, a mucopolysaccharide, a sugar, carries out the duty of reviving the follicle from its apparent state of atrophic quiescence by means of a nourishing action.

In the preferred embodiment, the composition further performs a reducing action which can advantageously be stabilised by means of the addition of antioxidant agents or free-radical scavengers such as, in particular, water-soluble cyclic hydroxylamines derived from N-piperidine, described in WO2005/084677 and U.S. Pat. No. 5,981,548, more particularly the compounds bis(1-oxyl (or 1-hydroxyl)-2,2,6,6-tetramethyl-4-piperidinyl)decandioate (hereinafter referred to as MP1002 and MP101). Said compounds are typically used in amounts of between 0.01 and 5 g/l.

The proteolytic agents induce a slight inflammatory component and gently remove (the concentrations of such enzymes are minimal in the composition) any obstacles present in inert follicles. For example, scar tissue reacts within a period of 20-40 days in culture with the composition being the object of the present invention, showing marked revitalisation and trophism of the follicles still present and actively producing strong and pigmented terminal hairs.

All the samples tested from patients with male, female and scar-tissue alopecia, remained alive in culture with active pilifer follicles for six months. All the biopsy samples treated show marked and constant repackaging of the hair strands, always caused by an order of topographic distribution and by trophic underlying tissue, free from lesions.

The composition being the object of the present invention has also been tested in vivo. The results obtained have been comparable to those obtained in vitro. On average, the first results have been observed two months after the start of treatment.

In one currently preferred embodiment, the composition being the object of the present invention comprises at least one proteolytic enzyme, such as for example papain, collagenase (preferably, type-IA, type-II, type-IV) serratiopeptidase, heparanase, DNAse, elastase, bromelain, bradykinase, Clostridium peptidase, enzymes expressed by Lactobacillus acidophilus, enzymes expressed by the genus Aspergillus, protease, alliinase, fibrinolysin, preferably in an alcoholic or hydroalcoholic solvent at a concentration of between 0.1 and 200 mg/l and at least one aminoacid, such as for example methionine, cystine, N-acetylcysteine, cysteine, glycine, leucine, proline, glutamine, arginine, preferably at a concentration of between 0.1 and 10% by weight with reference to the volume of the composition.

More preferably, the composition further comprises at least one peptide, preferably a reducing peptide, particularly an oligopeptide or a tripeptide, for example selected from glutathione, collagen, elastin and wheat extract.

More preferably, the composition further comprises a sugary solution, for example based on glucose, sucrose, glucans, mannans, glucomannans, fucose, fructose, heparan sulphates, pectins and starches, the alcohol derivatives thereof or mixtures thereof.

Even more preferably, the composition further comprises at least one mucopolysaccharide, such as for example hyaluronic acid and condroitin sulphates. As vitamins for example, retinoic acid, retinol, ascorbic acid, pantothenic acid, biotin or vitamin factors, such as for example inositol may be used; vitamins with reducing action, particularly ascorbic acid, are preferred.

The composition may furthermore comprise a so-called “penetration accelerator”. By the term “penetration accelerator” is meant a solution/compound capable of improving absorption; in particular, preferred compounds are those capable of improving the bioavailability of the composition, improving the vascularisation by means of increasing peripheral microcirculation and increased vasal permeability [7]. An example of a penetration accelerator is minoxidil or transcutol CG (ethoxydiglycol), liposomal vehicles, micellar vehicles, alcoholic and hydroalcoholic solutions, and mixtures thereof.

Preferably, the composition according to the present invention further comprises an aloe species extract.

Even more preferred are compositions comprising papain, collagenase, serratiopeptidase and/or elastase and mixtures thereof (0.1-200 g/l) as a proteolytic enzyme, in association with a mixture of aminoacids comprising methionine, cystine, acetylcysteine, glycine, leucine, proline and glutamine (preferred total concentration, 0.1-10% by weight with reference to volume) and with ascorbic acid, optionally in combination with one or more other vitamins selected from retinol, pantothenic acid and biotin (preferred total concentration 0.1-10% by weight/volume).

In a second embodiment, the present invention relates to a product comprising, as a combined preparation, at least one proteolytic enzyme and at least one aminoacid and/or one vitamin or vitamin factor and optionally one nucleotide or nucleoside for simultaneous, separate or sequential use in the recovery of original trophism and pigmentation and for stimulating the growth of the integumentary apparatus in mammals. In particular, the product may comprise two or more combined preparations, comprising at least one proteolytic enzyme and at least one component selected from an aminoacid, a nucleotide or nucleoside, a sugar, a vitamin or a mucopolysaccharide, and optionally one penetration accelerator, in various combinations.

The combined preparations may be administered by various routes, such as for example orally, parenterally, by endocavital surgery, topically. By way of example, a first preparation may be prepared for topical application onto the integumentary tissue of interest and comprise at least one proteolytic enzyme and minoxidil or transcutol CG (ethoxydiglycol) and hyaluronic acid and at least one sugar. The second preparation may be prepared for oral or parenteral administration and may comprise at least one aminoacid and at least one vitamin and optionally at least one sugar, such as for example glucose.

In one alternative form, a first preparation may be prepared for topical application to the integumentary tissue of interest and comprise at least one of the aminoacids, a vitamin or vitamin factor and optionally a nucleotide or a nucleoside and a peptide. The second preparation may be prepared for oral or parenteral administration and may comprise at least one proteolytic enzyme. The second preparation may further comprise a mucopolysaccharide.

The composition has been prepared in two different final concentrations with identical formulations:
Composition 1× (one times) for in vitro use, diluted in a solution of 0.2% w/v minoxidil, to give one litre of solution, or in a hydroalcoholic solution containing 5% transcutol CG (ethoxydiglycol) and 0.2% lauric acid, to give one litre of solution.
Composition 10× (ten times) for in vivo use, diluted in a solution of 0.2% w/v minoxidil, to give one litre of solution, or in 5% w/v transcutol CG (ethoxydiglycol) and 0.2% w/v lauric acid, to give one litre of solution.

The composition being the object of the present invention (known by the acronym FORM-XE) has also been developed to be used in cases of androgenic alopecia and refractory alopecia with a slow response (six months) to the FORM-XE composition, accelerating the hair regeneration process.

Said second composition (known by the acronym FORM-XE PLUS) acts by means of an inhibitor of 5-alpha-reductase, such as arginine or lauric acid (LAURIC ACID: formula: C₁₂H₂₄O, structure: CH₃(CH₂)₁₉ COOH, IUPAC: dodecanoic acid, synonym: laurostearic acid) and by means of the antioxidant support provided by glutathione or the stabilising and reducing support provided by hydroxylamine compounds derived from N-piperidine, previously cited in the dose range of between 0.01 g/l and 5 g/l.

### Composition for In Vitro Use

The composition being the object of the present invention for in vitro use, has been prepared using the substances in the quantities indicated in table 1.

TABLE 1

| Substance | mg/L |
|---|---|
| Methionine | 49.48 |
| Cystine | 51.29 |
| N-acetylcysteine | 10.00 |
| Cysteine | 35.12 |
| Glycine | 43.71 |
| Leucine | 159.05 |
| Proline | 46.75 |
| Glutamine | 450.00 |
| Biotin | 0.50 |
| Pantothenic acid | 2.50 |
| L-Ascorbic acid | 150.00 |
| Retinol | 0.015 |
| Papain | 2.00 |
| Hyaluronic acid | 5.00 |
| Glucose | 20.000.00 |

The substances have been weighed-out as required for the formula and the prepared dry ingredients have been diluted in a solution of 0.2% minoxidil to give one litre of solution.

The solution of 0.2% minoxidil has been prepared as follows: for one litre of solution, 2 g of powdered minoxidil sulphate, 540 g of absolute ethyl alcohol, 260 g of purified water and 200 g of propylene glycol have been used. The individual components have been mixed at room temperature in a glass container protected from light.

One variant of the composition being the object of the present invention further comprises 200 mg/ [symbol] arginine and 50 mg/ [symbol] glutathione.

A further variant of the composition being the object of the present invention further comprises substituting the 20% w/v propylene glycol with 5% transcutol CG (ethoxydiglycol), 0.20% w/v lauric acid and MP1001 and MP1002 in the dose range between 0.01 g/ [symbol] and 5 g/ [symbol] .

### Composition for In Vivo Use

The composition being the object of the present invention for in vivo use, has been prepared using the substances in the quantities indicated in table 2.

TABLE 2

| Substance | mg/L |
|---|---|
| Methionine | 494.8 |
| Cystine | 512.9 |
| N-acetylcysteine | 100.0 |
| Cysteine | 351.2 |
| Glycine | 437.1 |
| Leucine | 1590.5 |
| Proline | 467.5 |
| Glutamine | 4500.0 |
| Biotin | 5.0 |
| Pantothenic acid | 25.0 |
| L-Ascorbic acid | 1500.0 |
| Retinol | 0.15 |
| Papain | 20.0 |
| Hyaluronic acid | 50.0 |
| Glucose | 200.000.0 |

The substances have been weighed-out as required for the formula and the prepared dry ingredients have been diluted in a solution of 0.2% minoxidil to give one litre of solution.

One variant of the composition being the object of the present invention further comprises 2000 mg/ [symbol] arginine and 500 mg/ [symbol] glutathione.

A further variant of the composition being the object of the present invention further comprises substituting the 20% w/v propylene glycol with 5% w/v transcutol CG (ethoxydiglycol), 0.20% w/v lauric acid and water-soluble MP1001 and MP1002 in the dose range between 0.01 g/ [symbol] and 5 g/ [symbol] .

### Biopsies

All samples (human scalp biopsies) have been washed three times in isotonic saline containing antibiotics (100 units/ml of penicillin+100 ug/ml streptomycin+160 mg/ [symbol] gentamycin) for 10 minutes at room temperature.

The biopsy samples have then been dissected into three parts (two controls and one sample to be treated, for each patient) and suspended in a solution of FORM-XE with final concentration of 1× in 15 cm plates (Lab-Tek chamber slides, Nunc, Kamstrup, Denmark).

Two types of controls have been prepared: one negative control (1) treated only with isotonic saline and antibiotics (as described above), and one negative control (2) treated with cell culture medium:
1. the control biopsy samples have been suspended in isotonic saline in 15 cm plates (Lab-Tek chamber slides, Nunc, Kamstrup, Denmark).
2. The control biopsy samples have been placed in 15 cm plates (Lab-Tek chamber slides, Nunc, Kamstrup, Denmark) in RPMI 1640 medium supplemented with:
   10% FCS (Celbio, Milan, Italy)
   100 units/ml penicillin
   100 ug/ml streptomycin
   80 mg/L gentamycin (Schering-Plough, Italy)
   2 mM L-glutamine (Life Technologies)

All samples have been incubated in a Heraeus thermostatically controlled incubator at a temperature of 37° C. in an atmosphere containing a constant flow of 8% CO₂ (v/v in air).

Every 21 days in culture, the study culture supernatants have been collected and stored at −80° C. for the following laboratory analyses: total NO, nitrites and nitrates concentrations.

### Immunofluorescence Protocol

After three washes for 10 minutes at room temperature in PBS (pH 7.4), the samples have been resuspended for one hour at room temperature in a fixing solution, containing 4% paraformaldehyde in RPMI 1640 at pH 7.4. After inclusion in paraffin, the samples have been thin sectioned and placed on slides. The thin sections have been stained with hematoxylin/eosin, anti-cytokeratin 10 monoclonal antibodies (Santa Cruz Biotechnology, California, USA), anti-cytokeratin 11 monoclonal antibodies (Santa Cruz Biotechnology, California, USA). Specific controls with the corresponding isotypes have been devised for each monoclonal antibody (Santa Cruz Biotechnology, California, USA). All the samples on slides have been observed by light microscopy after sealing with moviol and cover slips.

### Measurement of NITRITES (NO₂⁻) and NITRATES (NO₃⁻)

Since the majority of the nitrogen monoxide (NO), produced by the NO synthetase enzymes, is oxidised to nitrites and nitrates, the concentration of said anions has been used as a quantitative assay for NO production. This method is based on the enzymatic conversion of the nitrates to nitrites through the use of the enzyme nitrate reductase.

The nitrites are subsequently identified spectrophotometrically by means of a colourimetric reaction which, in the presence of Griess reagent (naphthylethylenediamine dihydrochloride in 2N HCl and sulphanilamide in 2N HCl [Gross et al., 1991]), gives an azo-derivative as a final product which absorbs light at 540 nm. The NO concentration is calculated indirectly by measuring the levels of both nitrites and nitrates separately. This method allows evaluation of the quantity of endogenous nitrites present in the samples, and then subtracting it from the total values obtained.

### Measuring Endogenous Nitrites in the Samples

Samples of 100 μl of supernatant have been added to 100 μl of Griess reagent, and after incubating for 10 minutes the absorption of the solution has been read at 540 nm using a Packard model EL340 microplate reader. Fresh medium has been used for the blank, to be subtracted from each sample, and a calibration curve with known concentrations of sodium nitrite used as reference. The nitrite concentration has been expressed as μmoles of nitrite per mL.

Measuring Total Nitrites in the Samples Following Conversion of the Nitrates into Nitrites

Samples of 50 μl of supernatant have been added to 25 μl of nitrate reductase and 25 μl of NADH in 96 well plates, and left to incubate for 30 minutes at 37° C. Afterwards, 100 μl of Griess reagent have been added, and after incubating for 10 minutes, the absorbance of the solution has been read at 540 nm using a Packard model EL340 microplate reader. Fresh medium has been used for the blank, to be subtracted from each sample, and a calibration curve with known concentrations of sodium nitrate used as reference. The nitrite concentration has been expressed as μmoles of nitrite per mL.

### Determination of the Concentration of Nitrates in the Samples

In order to obtain the concentration of nitrates it is necessary to subtract the value obtained from the method for measuring endogenous nitrites from that obtained from the method for measuring total nitrites (after complete conversion of the nitrates to nitrites).

Measurement of the nitrites and nitrates has been performed using a kit: Nitric Oxide (NO₂⁻/NO₃⁻) Assay, cat. N^{o} DE1500, (R&D Systems Inc., Minneapolis Minn. 55413, USA).

### Measurement of Total Nitrogen Monoxide (NO)

This method is based on the enzymatic conversion of the nitrates to nitrites through the use of the enzyme nitrate reductase. The nitrites obtained react with the Griess reagent (naphthylethylenediamine dihydrochloride in 2N HCl and sulphanilamide in 2N HCl [Gross et al., 1991] giving an azo-derivative as final product which can be read optically at 540 nm. The quantity of NO is calculated indirectly based on the concentration of nitrites obtained from the total conversion of the nitrates into nitrites.

For the measurements, 50 μl samples of supernatant have been removed and added to 25 μl of nitrate reductase and 25 μl of NADH in 96 well plates, and left to incubate for 30 minutes at 37° C. Afterwards, 100 μl of Griess reagent have been added, and after incubating for 10 minutes, the absorbance of the solution has been read at 540 nm using a Packard model EL340 microplate reader. Fresh medium has been used for the blank, to be subtracted from each sample, and a calibration curve with known concentrations of sodium nitrate used as reference. The nitrite concentration has been expressed as μmoles of nitrite per mL.

Total NO measurements have been performed using the kit: Total Nitric Oxide Assay, cat. N^{o} DE1600, (R&D Systems Inc., Minneapolis Minn. 55413, USA).

### Western Blotting for Cytokeratins

The biopsy samples, suspended in lysis buffer (1% SDS, 30 mM Tris pH 6.8, 5% glycerol) to which protease inhibitors (Protease Inhibitor Cocktail, Calbiochem, San Diego, Calif.) have been added, have been homogenised and then the samples incubated for 30 minutes at 4° C. The lysates obtained have been centrifuged at 12,000 rpm for 20 minutes at 4° C. and the supernatants collected; the protein concentrations of the samples have been measured using the Bio-Rad method (Benchmark Plus assay, Bio-Rad). Prior to electrophoresis, the samples have been boiled for 5 minutes in the presence of beta-mercaptoethanol and bromophenol blue. The samples have been subjected to electrophoresis on a 12% gel (SDS-PAGE) and then transferred onto a PVDF membrane (Perkin Elmer Inc.). The membranes have been saturated with methanol at room temperature and subsequently incubated with the following primary antibodies diluted in PBS with 5% skimmed milk powder: anti-cytokeratin 14 at a dilution of 1:500 (Santa Cruz Biotechnologies Inc., Santa Cruz, Calif., USA), anti-cytokeratin 18 at a dilution of 1:500 (Santa Cruz Biotechnologies Inc., Santa Cruz, Calif., USA) and anti-cytokeratin 19 at a dilution of 1:500 (Santa Cruz Biotechnologies Inc., Santa Cruz, Calif., USA) overnight at 4° C. After washing five times, the membranes have been incubated with the corresponding secondary antibodies (1:1000) conjugated to horseradish peroxidase (HRP, Santa Cruz Biotechnologies Inc., Santa Cruz, Calif., USA) for 1 hour at room temperature. The corresponding bands have been revealed using chemiluminescence liquid (Super Signal Western Pico solution, Pierce Biotechnology Inc., Rockford, Ill., USA) and captured using photographic film.

### EXAMPLES

### Six Months Incubation

All the controls relevant to type 1 suspended only in isotonic saline, as described in materials and methods, quickly undergo a rapidly progressive necrotic process. They will thus be eliminated at day 15 of the study.

### Example 1

### Light Microscopy

These results show regenerating scalp that is free from alopecic disorders with good distribution of the normal stages of regrowth.

The results observed for expression of cytokeratins 10 and 11 and histological staining of the prepared biopsy samples with hematoxylin/eosin (used for observing follicle viability) are reported in table 3 and have been expressed on a quantitative scale.

From analysis of the results presented in Table 3 light microscopy (staining with eosin and hematoxylin) shows a marked increase in the number of follicles in the samples treated for six months with the composition being the object of the invention FORM-XE with respect to untreated controls. Furthermore, all the follicles appear trophic, viable and active in the samples treated for six months with the composition being the object of the invention FORM-XE with respect to controls, where atrophy or hypotrophism of the follicle itself is observed. Finally, a clear predominance of cytokeratins 10 and 11, typical of normal integumentary tissues with active and viable follicles, is observed in the samples treated for six months with the composition being the object of the invention FORM-XE with respect to untreated controls [8].

TABLE 3

| | | Treated | | Treated | | Treated |
|---|---|---|---|---|---|---|
| | Scar | scar | Male pt | male pt | Female | female |
| | tissue | tissue | scalp | scalp | pt scalp | pt scalp |
| Markers | control | sample | control | sample | control | sample |
| Cytokeratin | + | ++++ | ++ | ++++ | ++ | ++++ |
| 10 | | | | | | |
| Cytokeratin | + | ++++ | ++ | ++++ | ++ | ++++ |
| 11 | | | | | | |
| Eosin/ | ++ | +++ | ++ | ++++ | ++ | ++++ |
| Hematoxylin | | | | | | |

Legend
−−−−− = absence of any fluorescence
+ = low fluorescence per optic field
++ = medium fluorescence per optic field
+++ = high fluorescence per optic field
++++ = very high fluorescence per optic field
pt = patient

### Example 2

### Western Blotting

The samples have been subjected to phenotypic analyses by Western Blotting for the markers cytokeratin 14, cytokeratin 18 and cytokeratin 19, as discussed below.

The results show high positivity for cytokeratin 14, cytokeratin 18, cytokeratin 19 in the treated samples, and particularly in the scar tissue samples treated for six months in vitro with the composition being the object of the present invention, FORM-XE, with respect to only slight positivity for the production of cytokeratin 14, cytokeratin 18, cytokeratin 19 in the untreated control samples, as reported in Table 4. Cytokeratins 14, 18 and 19 are expressed in normal integumentary tissues with viable and active follicles during cell differentiation, pilifer follicular growth and controlled hair formation [8-9].

TABLE 4

| | | Treated | | Treated | Female | Treated |
|---|---|---|---|---|---|---|
| | Scar | scar | Male pt | male pt | pt | female |
| | tissue | tissue | scalp | scalp | scalp | pt scalp |
| Markers | control | sample | control | sample | control | sample |
| Cytokeratin | + | +++++ | + | ++++ | + | ++++ |
| 14 | | | | | | |
| Cytokeratin | + | +++++ | + | ++++ | + | ++++ |
| 18 | | | | | | |
| Cytokeratin | + | +++++ | + | ++++ | + | ++++ |
| 19 | | | | | | |

Legend
−−− = absence of any bands
−/+ = slight presence of a band
+ = thin band present
++ = medium band present
+++ = broad band present
++++ = high band present
+++++ = abundant band present
pt = patient

### Example 3

### Measurement of Nitrites, Nitrates and Total NO

TABLE 5

| | | Endogenous | | |
|---|---|---|---|---|
| | Total NO | nitrites | Nitrates | Signif. |
| SAMPLES | (μmol/L) | (μmol/L) | (μmol/L) | p value |
| Scar tissue treated | 202.16 | 4.51 | 197.65 | 0.001 |
| sample | | | | |
| Control 2 scar tissue | 13.76 | 9.16 | 4.60 | |
| at 1 month | | | | |
| Control 2 scar tissue | 14.96 | 7.77 | 7.19 | |
| at 2 months | | | | |
| Control 2 scar tissue | 12.96 | 8.70 | 4.26 | |
| at 3 months | | | | |
| Control 2 scar tissue | 9.36 | 4.98 | 4.38 | |
| at 6 months | | | | |
| Treated male pt sample | 180.16 | 2.65 | 177.51 | 0.001 |
| Control 2 young male pt | 12.16 | 6.37 | 5.79 | |
| at 1 month | | | | |
| Control 2 young male pt | 13.36 | 7.77 | 5.59 | |
| at 2 months | | | | |
| Control 2 young male pt | 14.56 | 8.70 | 5.86 | |
| at 3 months | | | | |
| Control 2 young male pt | 14.16 | 7.30 | 6.86 | |
| at 6 months | | | | |
| Older male patient | 225.76 | 0.79 | 224.97 | 0.001 |
| treated sample | | | | |
| Control 2 older male pt | 6.16 | 3.12 | 3.04 | |
| at 1 month | | | | |
| Control 2 older male pt | 13.36 | 8.70 | 4.66 | |
| at 2 months | | | | |
| Control 2 older male pt | 9.36 | 4.51 | 4.85 | |
| at 3 months | | | | |
| Control 2 older male pt | 11.36 | 7.77 | 3.59 | |
| at 6 months | | | | |
| Treated female pt | 187.36 | 1.72 | 185.64 | 0.001 |
| sample | | | | |
| Control 2 female pt at | 8.96 | 4.51 | 4.45 | |
| 1 month | | | | |
| Control 2 female pt at | 10.96 | 6.84 | 4.12 | |
| 2 months | | | | |
| Control 2 female pt at | 6.56 | 3.12 | 3.44 | |
| 3 months | | | | |
| Control 2 female pt at | 9.36 | 5.91 | 3.45 | |
| 6 months | | | | |
| Minimum detectable | 1.35 | 0.22 | 0.54 | |
| limit: | | | | |

pt = patient

Nitrogen oxide (NO), known incorrectly as nitric oxide, is a nitrogen-centred free-radical reactive chemical species. NO is transformed into a series of derivatives, such as nitrites and nitrates, which accumulate in a manner depending on the amounts of primary mediator produced in the blood and other extracellular fluids which are then removed from the body through the urine; plasma and urine levels of nitrites/nitrates correlate rather well in vivo with “endogenous” NO production, even following special treatments [10].

Thus overall, the experimental data analysed herein show that composition FORM-XE being the object of the invention, used in vitro can improve certain physio-pathological conditions through increased endogenous NO synthesis, as demonstrated by the corresponding increase in its catabolites, nitrites and nitrates. NO, and the derived nitrites and nitrates appear to assume the role of biological mediators which participate indirectly in the regulation of proliferation and differentiation of integumentary structures, specifically in the cyclic alterations affecting the pilifer follicle [11-12].

Analysis of the results presented in Table 5 shows a significant increase in the concentrations of nitrites and nitrates and, therefore, in the production of NO in samples treated in vitro with the composition being the object of the invention FORM-XE, typical of normal integumentary tissues with viable and active follicles.

Naturally, the details and forms of embodiment may be altered extensively with respect to the details described and illustrated without departing from the scope of protection of the present invention, as defined in the appended claims.

### Bibliography

1. Comaish S. “Metabolic disorders and hair growth” Br. J. Dermatol. 1971; 84:83.
2. Jeanmougin M. “Patologia dei capelli e del cuoio capelluto”, edizione italiana, Milano, CE.D.RI.M. S.r.l., 1991:9-12.
3. Tosti A, Peluso A. M., Piraccini B. M.: “Le malattie dei capelli e del cuoio capelluto” BIBLIOTECNE Milano, 1996:16.
4. Kossard S. “Postmenopausal frontal fibrosing alopecia: Scarring alopecia in a pattern distribution” Arch. Dermatol. 1994;130:770-774.
5. Schmidt J. B. “Hormonal basis of male and female androgenetic alopecia: Clinical relevance”. Skin Pharmacol. 1994;7:61-66.
6. Robinson M, Reynolds A J, Gharzi A, Jahoda C A. In vivo induction of hair growth by dermal cells isolated from hair follicles after extended organ culture. J. Invest. Dermatol. 2001 September; 117(3):596-604.
7. Shupack J L, Kassimir J J, Thirumoorthy T, Reed M L, Jondreau L. Dose-response study of topical minoxidil in male pattern alopecia. J. Am. Acad. Dermatol. 1987 March;16(3 Pt 2):673-6.
8. Limat A, Breitkreutz D, Stark H J, Hunziker T, Thikoetter G, Noser F, Fusenig N E. Experimental modulation of the differentiated phenotype of keratinocytes from epidermis and hair follicle outer root sheath and matrix cells. Ann. N. Y. Acad. Sci. 1991 December 26;642:125-46; discussion 146-7.
9. Bates E J, Penno N M, Hynd P I. Wool follicle matrix cells: culture conditions and keratin expression in vitro. Br. J. Dermatol. 1999 February; 140(2):216-25.
10. Fujiwara N, Osanai T, Kamada T, Katoh T, Takahashi K, Okumura K. Study on the relationship between plasma nitrite and nitrate level and salt sensitivity in human hypertension modulation of nitric oxide synthesis by salt intake. Circulation. 2000. 101:856-861.
11. Wu G, Meininger C J. Arginine nutrition and cardiovascular function. J. Nutr. 2000. 130: 2626-2629.
12. Oro A E, Higgins K. Hair cycle regulation of Hedgehog signal reception. Dev. Biol. 2003 March 15; 255(2): 238-48].

1. A composition useful for recovering original trophism and pigmentation and for stimulation of growth of the integumentary apparatus comprising at least one proteolytic enzyme and at least one member selected from an aminoacid and a vitamin or vitamin factor and mixtures thereof.

2. The composition according claim 1, characterised in that said at least one proteolytic enzyme is selected from papain, collagenase (preferably type-IA, type-II, type-IV) serratiopeptidase, heparanase, DNAse, elastase, bromelain, bradykinase, Clostridium peptidase, enzymes expressed by Lactobacillus acidophilus, enzymes expressed by the genus Aspergillus, proteases, alliinases, fibrinolysin and mixtures thereof.

3. The composition according to claims 1 or 2, characterised in that said at least one aminoacid is selected from methionine, cystine, N-acetylcysteine, cysteine, glycine, leucine, proline, glutamine, arginine and mixtures thereof.

4. The composition according to claim 3, characterised in that said at least one aminoacid is selected from cystine, cysteine and N-acetylcysteine and mixtures thereof.

5. The composition according to any preceding claim, characterised in that the composition further comprises at least one peptide, preferably an oligopeptide, even more preferably a tripeptide.

6. The composition according to claim 5, characterised in that said at least one peptide is selected from glutathione, collagen, elastin, wheat extract and mixtures thereof.

7. The composition according to any preceding claim, characterised in that the composition further comprises at least one sugar, preferably a monosaccharide or a polysaccharide, the alcohol derivatives thereof and mixtures thereof.

8. The composition according to claim 7, characterised in that said sugar is selected from glucose, sucrose, glucans, mannans, glucomannans, fucose, fructose, heparan sulphates, pectins, starches and mixtures thereof.

9. The composition according to any preceding claim, characterised in that the composition further comprises at least one mucopolysaccharide.

10. The composition according to claim 9, characterised in that said mucopolysaccharide is selected from hyaluronic acid, condroitin sulphates and mixtures thereof.

11. The composition according to any preceding claim, characterised in that the composition further comprises at least one nucleotide or nucleoside.

12. The composition according to any preceding claim, characterised in that said at least one vitamin is selected from retinoic acid, retinol, ascorbic acid, pantothenic acid and biotin and mixtures thereof.

13. The composition according to any of the claims 1 to 11, characterised in that said at least one vitamin factor is constituted by inositol.

14. The composition according to any preceding claim, characterised in that the composition further comprises a penetration accelerator, preferably minoxidil or transcutol CG (ethoxydiglycol), liposomal carriers, micellar carriers, alcoholic solutions, hydroalcoholic solutions or mixtures thereof.

15. The composition according to any preceding claim, characterised in that said at least one proteolytic enzyme is present in a quantity expressed as weight per volume with respect to the total volume of the composition, comprised of between 0.01 ng/L and 200 g/L.

16. The composition according to claim 15, characterised in that said at least one proteolytic enzyme is present in a quantity expressed as a percentage weight with respect to the total volume of the composition, comprised of between 0.01 mg/L and 200 mg/L.

17. The composition according to any preceding claim, characterised in that said at least one aminoacid is present in a quantity expressed as percentage weight with respect to the total volume of the composition, comprised of between 0.001% and 99.9%.

18. The composition according to claim 17, characterised in that said at least one aminoacid is present in a quantity expressed as a percentage weight with respect to the total volume of the composition, comprised of between 0.1% and 10%.

19. The composition according to any preceding claim, characterised in that said at least one peptide is present in a quantity expressed as percentage weight with respect to the total volume of the composition, comprised of between 0.01% and 99.9%.

20. The composition according to claim 19, characterised in that said at least one peptide is present in a quantity expressed as a percentage weight with respect to the total volume of the composition, comprised of between 0.1% and 20%.

21. The composition according to any preceding claim, characterised in that said at least one sugar is present in a quantity expressed as percentage weight with respect to the total volume of the composition, comprised of between 0.01% and 99.9%.

22. The composition according to claim 21, characterised in that said at least one sugar is present in a quantity expressed as a percentage weight with respect to the total volume of the composition, comprised of between 0.1% and 20%.

23. The composition according to any preceding claim, characterised in that said mucopolysaccharide is present in a quantity expressed as weight with respect to the total volume of the composition, comprised of between 0.01 mg/L and 50 g/L.

24. The composition according to claim 23, characterised in that said mucopolysaccharide is present in a quantity expressed as weight with respect to the total volume of the composition, comprised of between 0.1 mg/L and 200 mg/L.

25. The composition according to any preceding claim, characterised in that said at least one vitamin or vitamin factor is present in a quantity expressed as percentage weight with respect to the total volume of the composition, comprised of between 0.01% and 99.9%.

26. The composition according to claim 25, characterised in that said at least one vitamin or vitamin factor is present in a quantity expressed as a percentage weight with respect to the total volume of the composition, comprised of between 0.01% and 10%.

27. The composition according to any preceding claim, characterised in that said penetration accelerator is present in a quantity expressed as percentage weight with respect to the total volume of the composition, comprised of between 0.01% and 99.9%.

28. The composition according to claim 27, characterised in that said penetration accelerator is present in a quantity expressed as a percentage weight with respect to the total volume of the composition, comprised of between 0.01% and 10% .

29. The composition according to any of the claims 1 to 27, characterised in that the composition comprises at least one aloe species extract.

30. The composition according to any of the claims 1 to 29, characterised in further comprising an inhibitor of 5-alpha reductase selected from arginine and lauric acid.

31. The composition according to claim 30, characterised in comprising lauric acid in quantities of between 0.2 and 10 g/l.

32. The composition according to any of the claims 1 to 31, characterised in comprising bis(1-oxyl, or (1-hydroxyl)-2,2,6,6-tetramethyl-4-piperidinyl)decandioate, preferably in a quantity of between 0.01 and 5 g/l.

33. The composition according to any of the claims 1 to 32, characterised in that the composition has a formulation capable of being administered topically, parenterally, by endocavital surgery, orally.

34. The use of a composition according to any of the claims 1 to 33 for the normalisation of the original trophism of the integumentary apparatus in mammals.

35. The use of a composition according to any of the claims 1 to 33 for restoring the original pigmentation of the integumentary apparatus in mammals.

36. The use of a composition according to any of the claims 1 to 33 for the stimulation of growth of the integumentary apparatus in mammals.

37. The use according to claim 36 for the cosmetic treatment of alopecia, defluvium or effluvium in mammals.

38. The use according to any of the claims 34 to 37, wherein the composition is administered topically, parenterally, by endocavital surgery, orally.

39. The use of at least one proteolytic enzyme selected from papain, collagenase (preferably type-IA, type-II, type-IV) serrathiopeptidase, heparanase, DNAse, elastase, bromelain, bradykinase, Clostridium peptidase, enzymes expressed by Lactobacillus acidophilus, enzymes expressed by the genus Aspergillus, proteases, alliinases, fibrinolysin for the normalisation of original trophism of the integumentary apparatus.

40. The use according to claim 39, characterised in that said at least one proteolytic enzyme is in association with at least one aminoacid or one vitamin or vitamin factor, and mixtures thereof.

41. The use according to claim 40, characterised in that said at least one aminoacid is selected from methionine, cystine, N-acetylcysteine, cysteine, glycine, leucine, proline, glutamine, arginine and mixtures thereof, preferably cystine, cysteine and N-acetylcysteine.

42. The use according to any of the claims 39 to 41, characterised in that said at least one proteolytic enzyme is in association with at least one sugar, preferably a monosaccharide or a polysaccharide, the alcohol derivatives thereof or mixtures thereof.

43. The use according to claim 42, characterised in that said sugar is selected from glucose, sucrose, glucans, mannans, glucomannans, fucose, fructose, heparan sulphates, pectins, starches.

44. The use according to any of the claims 39 to 43, characterised in that said at least one proteolytic enzyme is in association with at least one mucopolysaccharide, preferably hyaluronic acid, condroitin sulphates.

45. The use according to any of the claims 39 to 44, characterised in that said at least one proteolytic enzyme is in association with at least one vitamin, selected from retinoic acid, retinol, ascorbic acid, pantothenic acid and biotin.

46. The use according to any of the claims 39 to 45, characterised in that said at least one proteolytic enzyme is in association with at least one vitamin factor, preferably inositol.

47. The use according to any of the claims 39 to 46, characterised in that said at least one proteolytic enzyme is in association with a penetration accelerator, preferably minoxidil or transcutol CG (ethoxydiglycol), liposomal carriers, micellar carriers, alcoholic solutions, hydroalcoholic solutions or mixtures thereof.

48 . The use according to any of the claims 39 to 47, characterised in that said at least one proteolytic enzyme is in association with at least one aloe species extract.

49. A product comprising the components of a composition according to any of the claims 1 to 33 as a combined preparation for simultaneous, separate or sequential use, in the restoration of original pigmentation of the integumentary apparatus in mammals, or in the stimulation of growth of the integumentary apparatus in mammals, preferably hair/head hair.

50. The product according to claim 49, characterised in that a first preparation is in a form that can be administered topically, and a second preparation is in a form that can be administered orally, parenterally or by endocavital surgery, where said at least one first preparation comprises at least one proteolytic enzyme and said at least one second preparation comprises at least one of the aminoacids, a vitamin or vitamin factor or mixtures thereof.
